(19) United States
(12) Patent Application Publication
(10) Pub. No.: US 2021/0330911 A1
(43) Pub. Date: Oct. 28, 2021

(54) COLLAPSIBLE HEALTHCARE BARRIER DEVICE

(71) Applicant: Atlas ATC Enterprises LLC, Pflugerville, TX (US)

(72) Inventor: Andrew T. Crain, Pflugerville, TX (US)

(21) Appl. No.: 16/860,577

(22) Filed: Apr. 28, 2020

(51) Int. Cl. A61M 16/00; B65D 6/16

(52) U.S. Cl. CPC A61M 16/0087; A61M 2210/0606; A61M 2202/206; B65D 11/18

(57) ABSTRACT

A collapsible healthcare barrier device may be moveable between collapsed and erected positions. The device may include a left side panel, a front panel, a right side panel, and a top panel. The left side panel is hingedly connected to the left side of the front panel. The right side panel is hingedly connected to the right side of the front panel. The top panel is hingedly connected to the top of the front panel. The front panel includes at least one passageway through which a health care provider may extend his or her hands and arms to render care to a patient. The top panel may include a left lip member and a right lip member. A back flap may be hingedly connected to the rear of the top panel. The back flap may include a left wing member and a right wing member.

### BACKGROUND OF THE INVENTIONS

### 1. Field of the Inventions

The present inventions generally pertain to devices for use in hospital or other medical settings, and more particularly to devices that function to shield a health care giver from a patient, such as one having a contagious condition and/or emitting aerosolized contaminants or expectorants.

### 2. Description of the Related Art

The global coronavirus of 2020 has impacted human behavior in a vast variety of ways, including in the medical field. As hospitals have been overwhelmed with patients suffering from the coronavirus, health care professionals charged with the responsibility of treating and caring for these patients have had to adapt the way they provide care. In addition to the use of personal protection equipment (PPE), one way health care providers have done this is through the use of barriers positioned between themselves and their patients. One example of such a barrier is a four-sided box structure having a left side, a front side, a right side, and a top side. The box is made of transparent material. The front side includes two circular passageways. The box is positioned around the head and upper chest region of the patient, with the patient's head positioned adjacent the front side and two circular passageways. A health care giver stands near the patient's head with the front side of the box positioned between the health care giver and the patient. The health care giver then inserts his or her arms through the circular passageways to render care to the patient. One example of a procedure that can be performed using the box is an intubation. Examples of transparent health care barrier boxes of the type described above can be found at the following links: https://sites.google.com/view/aerosolbox/design; https://intubationbox.com/; https://www.nejm.org/doi/full/10.1056/NEJMc2007589 (New England Journal of Medicine Study); and https://www.vacep.org/intubationbox (Virginia College of Emergency Physicians).

It has been determined that the box structure as described above has shortcomings, including that it occupies too much valuable space when it is not in use and being stored, and another being that it is not easily portable. As will become apparent from the description and explanation set forth below, the present inventions overcome the above-described problems by providing collapsible box structures for use in shielding health care givers from patients while rendering care to them.

### SUMMARY OF THE INVENTIONS

Collapsible healthcare barrier devices are disclosed. In one aspect, a specific embodiment of the present inventions may be a health care barrier device comprising a left side panel having a front edge, a top edge, a bottom edge, a rear edge, an inner surface, and an outer surface; a front panel having a left edge, a top edge, a right edge, a bottom edge, at least one passageway, an inner surface, and an outer surface; a right side panel having a front edge, a top edge, a bottom edge, a rear edge, an inner surface, and an outer surface; a top panel having a left edge, a front edge, a right edge, and a rear edge; the front edge of the left side panel being hingedly connected to the left edge of the front panel; the front edge of the right side panel being hingedly connected to the right edge of the front panel; the top edge of the front panel being hingedly connected to the front edge of the top panel; and the left side panel, the front side panel, the right side panel, and the top panel being moveable between collapsed and erected positions. Another feature of this aspect of the present inventions may be that the device may further include a left hinge connecting the left side panel to the front panel; a right hinge connecting the right side panel to the front panel; and a top hinge connecting the front panel to the top panel. Another feature of this aspect of the present inventions may be that the device may further include a back flap having a top edge hingedly connected to the rear edge of the top panel, the back flap being moveable between a collapsed position and an erected position. Another feature of this aspect of the present inventions may be that the device may further include a rear hinge connecting the back flap to the top panel. Another feature of this aspect of the present inventions may be that the back flap may further include a left wing member extending from a left edge of the back flap and a right wing member extending from a right edge of the back flap. Another feature of this aspect of the present inventions may be that the left wing member and the right wing member may be disposed in generally perpendicular relationship with the back flap. Another feature of this aspect of the present inventions may be that the top panel may further include a left lip member extending from the left edge of the top panel, and a right lip member extending from the right edge of the top panel, the left lip member being positioned adjacent the top edge and the outer surface of the left side panel when the top panel and the left side panel are in their erected positions, the right lip member being positioned adjacent the top edge and the outer surface of the right side panel when the top panel and the right side panel are in their erected positions. Another feature of this aspect of the present inventions may be that the left lip member and the right lip member may be disposed in generally perpendicular relationship with the top panel.

In another aspect, a specific embodiment may include a health care barrier device comprising: a left side panel having a front edge, a top edge, a bottom edge, a rear edge, an inner surface, and an outer surface; a front panel having a left edge, a top edge, a right edge, a bottom edge, at least one passageway, an inner surface, and an outer surface; a right side panel having a front edge, a top edge, a bottom edge, a rear edge, an inner surface, and an outer surface; a top panel having a left edge, a front edge, a right edge, and a rear edge; the front edge of the left side panel being hingedly connected to the left edge of the front panel; the front edge of the right side panel being hingedly connected to the right edge of the front panel; the top edge of the front panel being hingedly connected to the front edge of the top panel; the left side panel, the front side panel, the right side panel, and the top panel being moveable between collapsed and erected positions; and, the top panel further including a left lip member extending from the left edge of the top panel, and a right lip member extending from the right edge of the top panel, the left lip member being positioned adjacent the top edge and the outer surface of the left side panel when the top panel and the left side panel are in their erected positions, the right lip member being positioned adjacent the top edge and the outer surface of the right side panel when the top panel and the right side panel are in their erected positions. Another feature of this aspect of the present inventions may be that the device may further include a left hinge connecting the left side panel to the front panel; a right hinge connecting the right side panel to the front panel; and a top hinge connecting the front panel to the top panel. Another feature of this aspect of the present inventions may be that the left lip member and the right lip member may be disposed in generally perpendicular relationship with the top panel. Another feature of this aspect of the present inventions may be that the device may further include a back flap having a top edge hingedly connected to the rear edge of the top panel, the back flap being moveable between a collapsed position and an erected position. Another feature of this aspect of the present inventions may be that the device may further include a rear hinge connecting the back flap to the top panel. Another feature of this aspect of the present inventions may be that the back flap may further include a left wing member extending from a left edge of the back flap and a right wing member extending from a right edge of the back flap, the left wing member being positioned adjacent an outer surface of the left lip member when the top panel, the left side panel, and the back flap are in their erected positions, and the right wing member being positioned adjacent an outer surface of the right lip member when the top panel, the right side panel, and the back flap are in their erected positions. Another feature of this aspect of the present inventions may be that the left wing member and the right wing member are disposed in generally perpendicular relationship with the back flap.

In another aspect, a specific embodiment may include a health care barrier device comprising: a left side panel having a front edge, a top edge, a bottom edge, a rear edge, an inner surface, and an outer surface; a front panel having a left edge, a top edge, a right edge, a bottom edge, at least one passageway, an inner surface, and an outer surface; a right side panel having a front edge, a top edge, a bottom edge, a rear edge, an inner surface, and an outer surface; a top panel having a left edge, a front edge, a right edge, and a rear edge; a back flap having a left edge, a top edge, and a right edge; the front edge of the left side panel being hingedly connected to the left edge of the front panel; the front edge of the right side panel being hingedly connected to the right edge of the front panel; the top edge of the front panel being hingedly connected to the front edge of the top panel; the top edge of the back flap being hingedly connected to the rear edge of the top panel, the left side panel, the front side panel, the right side panel, the top panel, and the back flap being moveable between collapsed and erected positions; and, the top panel further including a left lip member extending from the left edge of the top panel, and a right lip member extending from the right edge of the top panel, the left lip member being positioned adjacent the top edge and the outer surface of the left side panel when the top panel and the left side panel are in their erected positions, the right lip member being positioned adjacent the top edge and the outer surface of the right side panel when the top panel and the right side panel are in their erected positions. Another feature of this aspect of the present inventions may be that the device may further include a left hinge connecting the left side panel to the front panel; a right hinge connecting the right side panel to the front panel; a top hinge connecting the front panel to the top panel; and a rear hinge connecting the back flap to the top panel. Another feature of this aspect of the present inventions may be that the left lip member and the right lip member may be disposed in generally perpendicular relationship with the top panel. Another feature of this aspect of the present inventions may be that the back flap may further include a left wing member extending from a left edge of the back flap and a right wing member extending from a right edge of the back flap, the left wing member being positioned adjacent an outer surface of the left lip member when the top panel, the left side panel, and the back flap are in their erected positions, and the right wing member being positioned adjacent an outer surface of the right lip member when the top panel, the right side panel, and the back flap are in their erected positions. Another feature of this aspect of the present inventions may be that the left wing member and the right wing member may be disposed in generally perpendicular relationship with the back flap.

Other features, aspects and advantages of the present inventions will become apparent from the following discussion and detailed description.

### BRIEF DESCRIPTION OF THE DRAWINGS

FIG. 1 is a perspective view of a specific embodiment of a collapsible healthcare barrier device constructed in accordance with the present inventions, and shown in an erected position.

FIG. 2 is an elevation view showing the collapsible healthcare barrier device shown in FIG. 1, but in an expanded position.

FIG. 3 is a cross-sectional view taken along line 3-3 in FIG. 2, and showing a top view of a left side panel, a front panel, and a right side panel of a collapsible healthcare barrier device in its expanded position (but without a top panel or lid).

FIG. 4 is a top view similar to FIG. 3, except the left side panel and right side panel have been rotated into their erected positions.

FIG. 5 is a side view showing the cross section of a specific embodiment of a living hinge that may be used as part of the present inventions.

FIG. 6 is a perspective view showing a specific embodiment of a collapsible healthcare barrier device in its erected configuration and positioned over the head and chest of a patient lying on a hospital bed.

FIG. 7 is a perspective view similar to FIG. 6 but further showing a health care provider standing adjacent the patient's head and extending his or her hands and arms through the passageways in the front panel of the device to render care to the patient.

FIG. 8 is a side view of a specific embodiment of a collapsible healthcare barrier device shown in a collapsed position.

FIG. 9 is a top view similar to FIG. 3, but further showing the top panel in its erected position.

FIG. 10 is a top view similar to FIG. 4, but further showing the top panel in its erected position.

FIG. 11 is a side view from the left side of a collapsible healthcare barrier device when it is in its collapsed position.

FIG. 12 is a side view from the right side of a collapsible healthcare barrier device when it is in its collapsed position.

FIG. 13 is a perspective view showing a collapsible healthcare barrier device in the process of being moved from its collapsed position to its erected position.

FIG. 14 is a perspective view of the device shown in FIG. 13 with the right side panel, left side panel, and top panel in their erected positions, and the optional flap in the process of being rotated into its erected position.

FIG. 15 is a perspective view of the device shown in FIGS. 13 and 14, but with the optional flap rotated into its erected or closed position.

FIG. 16 is a perspective view of a specific embodiment of a health care barrier device which includes a single passageway in the front panel.

While the inventions will be described in connection with the preferred embodiments, it will be understood that the scope of protection is not intended to limit the inventions to those embodiments. On the contrary, the scope of protection is intended to cover all alternatives, modifications, and equivalents as may be included within the spirit and scope of the inventions as defined by the appended claims.

### DETAILED DESCRIPTION OF THE INVENTION

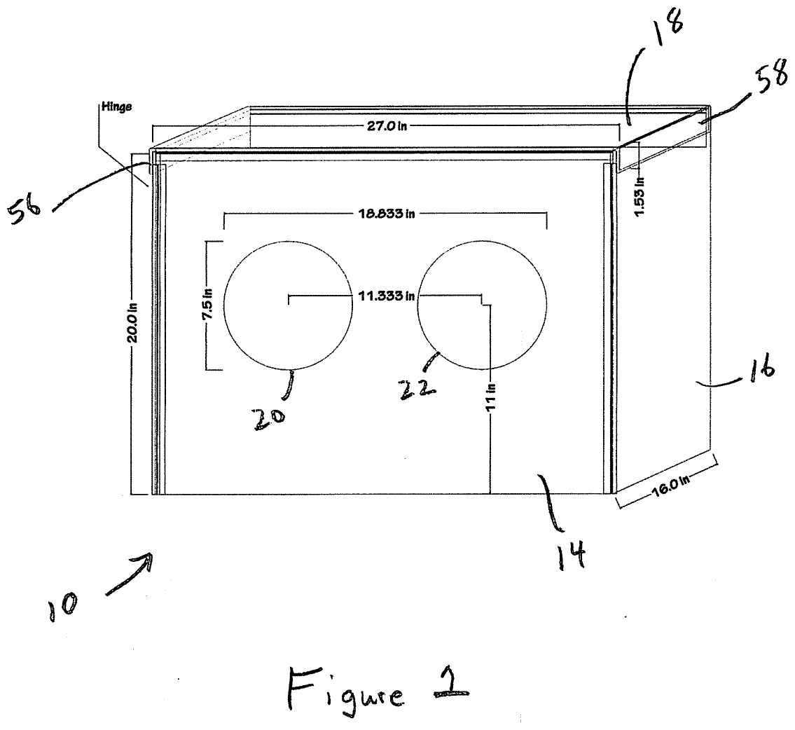

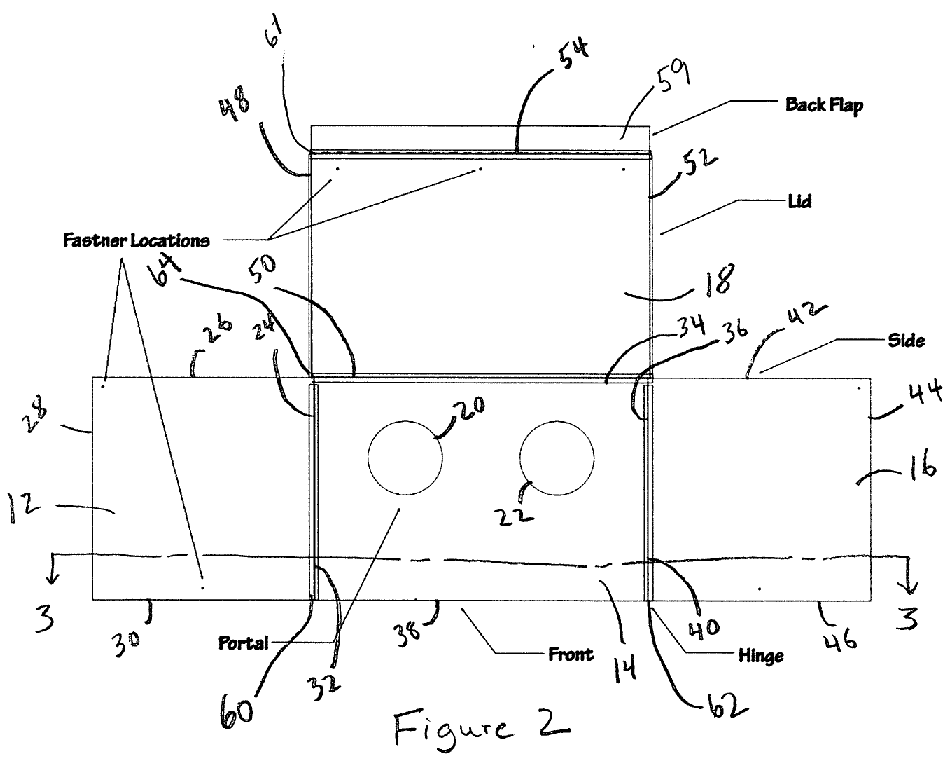

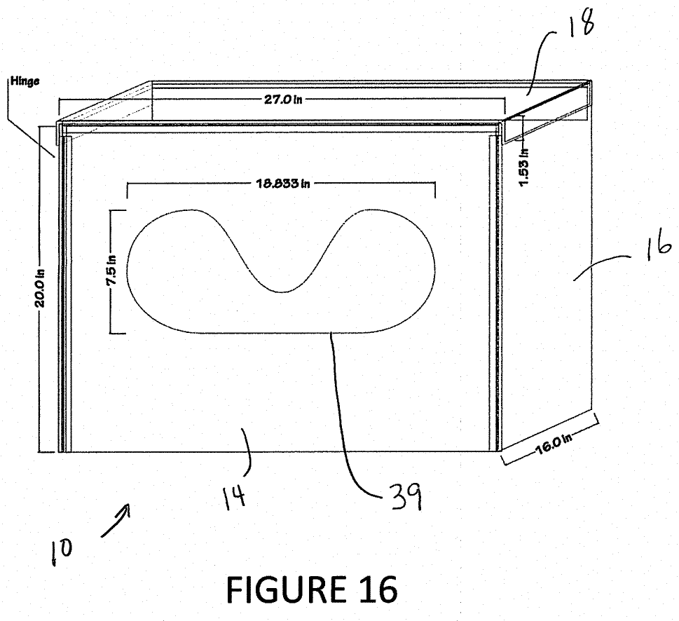

Referring to the drawings in detail, wherein like numerals denote identical elements throughout the several views, and referring initially to FIG. 1, there is shown a perspective view of a collapsible healthcare barrier device 10 in an erected or installed position. The device 10 is shown in FIG. 2 in an expanded position. The device 10 includes a left side panel 12, a front panel 14, a right side panel 16, and a top panel or lid 18. Each of the panels 12-18 is preferably made from a clear or transparent material, such as an acrylic or polycarbonate material, for example. In a specific embodiment, the thickness of the material may be 3/16 inches. The front panel 14 may include a first passageway 20 and a second passageway 22. In a specific embodiment, the first and second passageways 20 and 22 may be circular in shape. In a specific embodiment, the first and second passageways 20 and 22 may have a diameter of seven and one-half inches. In a specific embodiment, as shown in FIG. 16, the device 10 may include at least one passageway 39 through which a health care provider may extend both hands and arms to render care to a patient. In a specific embodiment, the dimensions of the left side panel 12 and the right side panel 16 may be 20 inches tall and 16 inches deep. In a specific embodiment, the dimensions of the front panel 14 may be 20 inches tall and 27 inches wide. In a specific embodiment, the dimensions of the top panel 18 may be 27 inches wide and 16 inches deep.

With reference to FIG. 2, the left side panel 12 includes a front edge 24, a top edge 26, a rear edge 28, and a bottom edge 30. The front panel 14 includes a left edge 32, a top edge 34, a right edge 36, and a bottom edge 38. The right side panel 16 includes a front edge 40, a top edge 42, a rear edge 44, and a bottom edge 46. The top panel 18 includes a left edge 48, a front edge 50, a right edge 52, and a rear edge 54.

In a specific embodiment, the top panel 18 may include a left lip member 56 extending downwardly from the left edge 48 of the top panel 18. In a specific embodiment, the left lip member 56 may be disposed in generally perpendicular relationship to the top panel 18. In a specific embodiment, the top panel 18 may include a right lip member 58 extending downwardly from the right edge 52 of the top panel 18. In a specific embodiment, the right lip member 58 may be disposed in generally perpendicular relationship to the top panel 18. In a specific embodiment, the left lip member 56 and the right lip member 58 may be formed as part of the top panel 18.

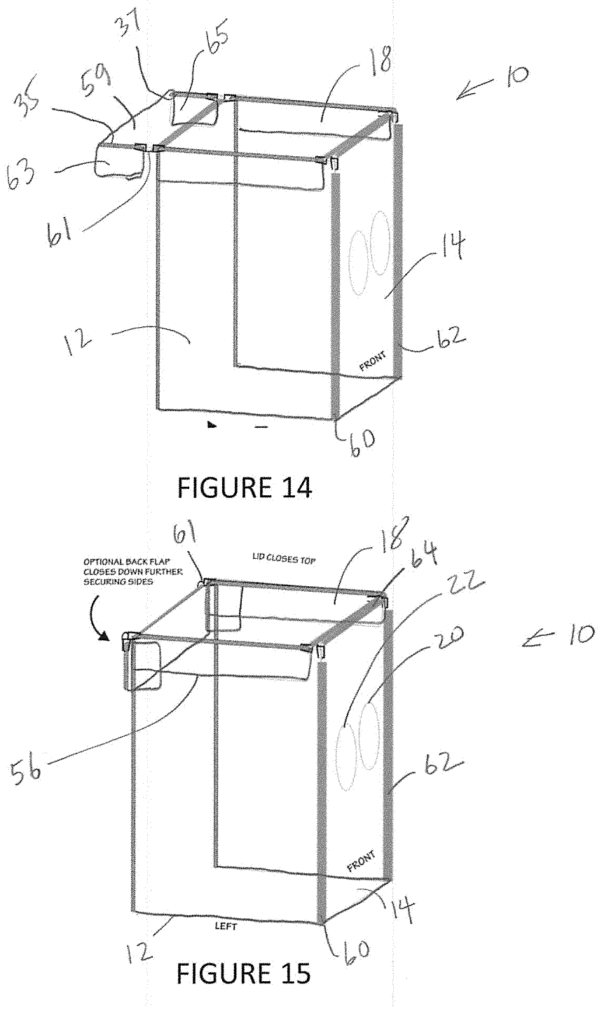

In a specific embodiment, the top panel 18 may include a back flap 59 hingedly connected by a rear hinge 61 to the rear edge 54 of the top panel 18. A back flap 59 may be included to provide additional stability to the collapsible healthcare barrier device 10 when in its erected position, and/or to reduce aerosolized contaminant spread from the upper back portion of the device 10. In a specific embodiment, as shown for example in FIG. 14, the back flap 59 may include a left wing member 63 extending from a left edge 35 of the back flap 59, and a right wing member 65 extending from a right edge 37 of the back flap 59. In a specific embodiment, the left wing member 63 and the right wing member 65 may be disposed in generally perpendicular relationship to the back flap 59. In a specific embodiment, the left wing member 63 and the right wing member 65 may be formed as part of the back flap 59. With reference to FIGS. 14 and 15, when the back flap is rotated downwardly from its position shown in FIG. 14 to its closed position shown in FIG. 15, the left wing member 63 will be positioned adjacent to, preferably in contact with, an outer surface 67 of the left lip member 56 on the top panel 18, and the right wing member 65 will be positioned adjacent to, preferably in contact with, an outer surface 69 of the right lip member 58 on the top panel 18. In a specific embodiment, the dimensions of the back flap 59 may be in the range of 26½ to 27½ inches wide, and two to four inches tall.

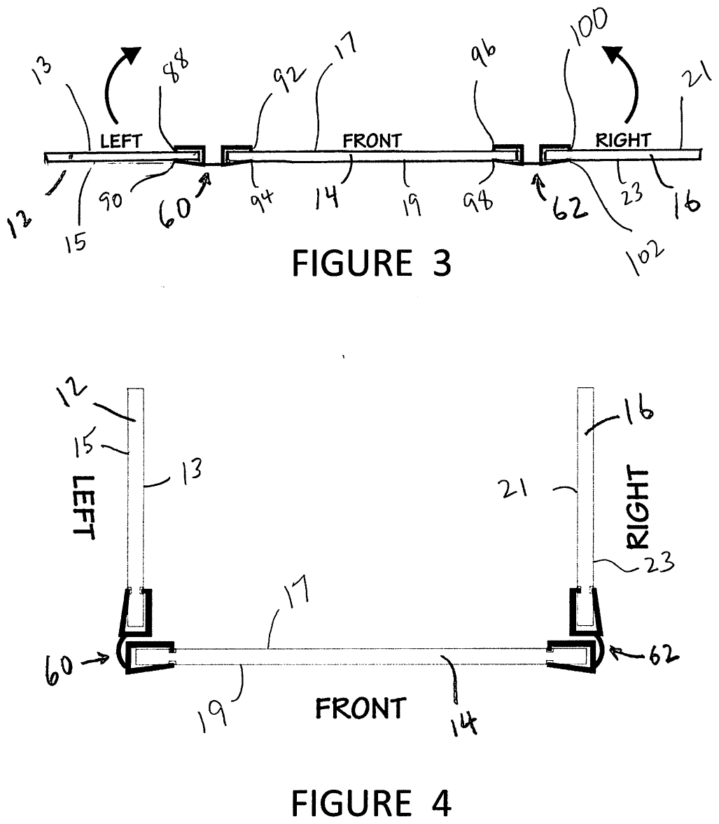

The front edge 24 of the left side panel 12 is hingedly connected by a left hinge 60 to the left edge 32 of the front panel 14. The front edge 40 of the right side panel 16 is hingedly connected by a right hinge 62 to the right edge 36 of the front panel 14. The top edge 34 of the front panel 14 is hingedly connected by a top hinge 64 to the front edge 50 of the top panel 18. In a specific embodiment, as shown in FIGS. 3 and 4, the hinges 60, 62, and 64 (and also the hinge 61 discussed below) may be living hinges of the type available from United States Plastics Corporation, of Lima, Ohio, as shown on its website at https://www.usplastic.com/catalog/item.aspx?itemid=35978&clickid=search (such as Item Number 91624, 3/16″ Living Hinge). The meaning of “living hinge” as used herein shall be a hinge of the general type shown on the foregoing webpage and in FIGS. 3-5.

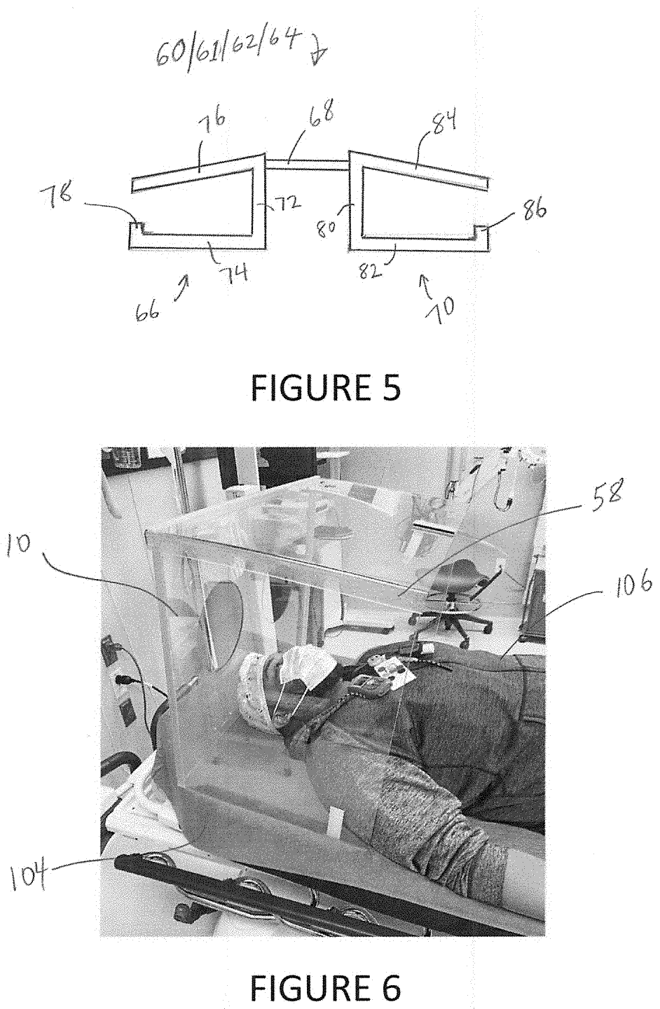

With reference to FIG. 5, an example of a living hinge 60/62/64 (and also the hinge 61 discussed below) is shown, which may include a left attachment member 66 connected by a central connector 68 to a right attachment member 70. The left attachment member 66 and the right attachment member 70 may be generally U-shaped structures that face away from each other. The left attachment member 66 includes a left base member 72, a left lower arm 74 extending away from a lower end of the left base member 72, and a left upper arm 76 extending away from an upper end of the left base member 72. A distal end of the left lower arm 74 includes a right upwardly extending lip 78. The right attachment member 70 includes a right base member 80, a right lower arm 82 extending away from a lower end of the right base member 80, and a right upper arm 84 extending away from an upper end of the right base member 80. A distal end of the right lower arm 82 includes a right upwardly extending lip 86. The central connector 68 may be connected between upper ends of the left base member 72 and the right base member 80.

Referring to FIG. 3, the left side panel 12, the front panel 14, and the right side panel 16 are shown in the expanded position. Referring now to FIG. 4, the left side panel 12 and the right side panel 16 have been rotated rearwardly into the erected or installed position. The top panel 18 is not shown in FIG. 3 or FIG. 4, but are shown in FIGS. 9 and 10. FIGS. 3 and 4 also illustrate the manner in which the living hinges 60 and 62 are secured to the left side panel 12, front panel 14, and the right panel 16 (which is the same way the top living hinge 64 is used to hingedly secure the top panel 18 to the front panel 14, and the way the rear hinge 61 is used to hingedly secure the back flap 59 to the rear of the top panel 18, as further discussed below).

With reference to FIG. 3, the left side panel 12 includes an inner longitudinal slot 88 on an inner surface 13 of the left side panel 12 and adjacent the front edge 24 of the left side panel 12. In a specific embodiment, the inner longitudinal slot 88 is generally parallel to the front edge 24 of the left side panel 12. The right lip 86 on the hinge 60 is engaged with the inner longitudinal slot 88 on the left side panel 12. The left side panel 12 includes an outer longitudinal slot 90 on an outer surface 15 of the left side panel 12 and adjacent the front edge 24 of the left side panel 12. In a specific embodiment, the outer longitudinal slot 90 is generally parallel to the front edge 24 of the left side panel 12. A distal end of the right upper arm 84 on the hinge 60 is engaged with the outer longitudinal slot 90 on the left side panel 12.

The front panel 14 includes a left inner longitudinal slot 92 on an inner surface 17 of the front panel 14 and adjacent the left edge 32 of the front panel 14. In a specific embodiment, the left inner longitudinal slot 92 is generally parallel to the left edge 32 of the front panel 14. The left lip 78 on the hinge 60 is engaged with the left inner longitudinal slot 92 on the front panel 14. The front panel 14 includes a left outer longitudinal slot 94 on an outer surface 19 of the front panel 14 and adjacent the left edge 32 of the front panel 14. In a specific embodiment, the left outer longitudinal slot 94 is generally parallel to the left edge 32 of the front panel 14. A distal end of the left upper arm 76 on the hinge 60 is engaged with the left outer longitudinal slot 94 on the front panel 14.

The front panel 14 includes a right inner longitudinal slot 96 on the inner surface 17 of the front panel 14 and adjacent the right edge 36 of the front panel 14. In a specific embodiment, the right inner longitudinal slot 96 is generally parallel to the right edge 36 of the front panel 14. The right lip 86 on the hinge 62 is engaged with the right inner longitudinal slot 96 on the front panel 14. The front panel 14 includes a right outer longitudinal slot 98 on the outer surface 19 of the front panel 14 and adjacent the right edge 36 of the front panel 14. In a specific embodiment, the right outer longitudinal slot 98 is generally parallel to the right edge 36 of the front panel 14. A distal end of the right upper arm 84 on the hinge 62 is engaged with the right outer longitudinal slot 98 on the front panel 14.

The right side panel 16 includes a left inner longitudinal slot 100 on an inner surface 21 of the right side panel 16 and adjacent the front edge 40 of the right side panel 16. In a specific embodiment, the left inner longitudinal slot 100 is generally parallel to the front edge 40 of the right side panel 16. The left lip 78 on the hinge 62 is engaged with the left inner longitudinal slot 100 on the right side panel 16. The right side panel 16 includes a left outer longitudinal slot 102 on an outer surface 23 of the right side panel 16 and adjacent the front edge 40 of the right side panel 16. In a specific embodiment, the left outer longitudinal slot 102 is generally parallel to the front edge 40 of the right side panel 16. A distal end of the left upper arm 76 on the hinge 62 is engaged with the left outer longitudinal slot 102 on the right side panel 16.

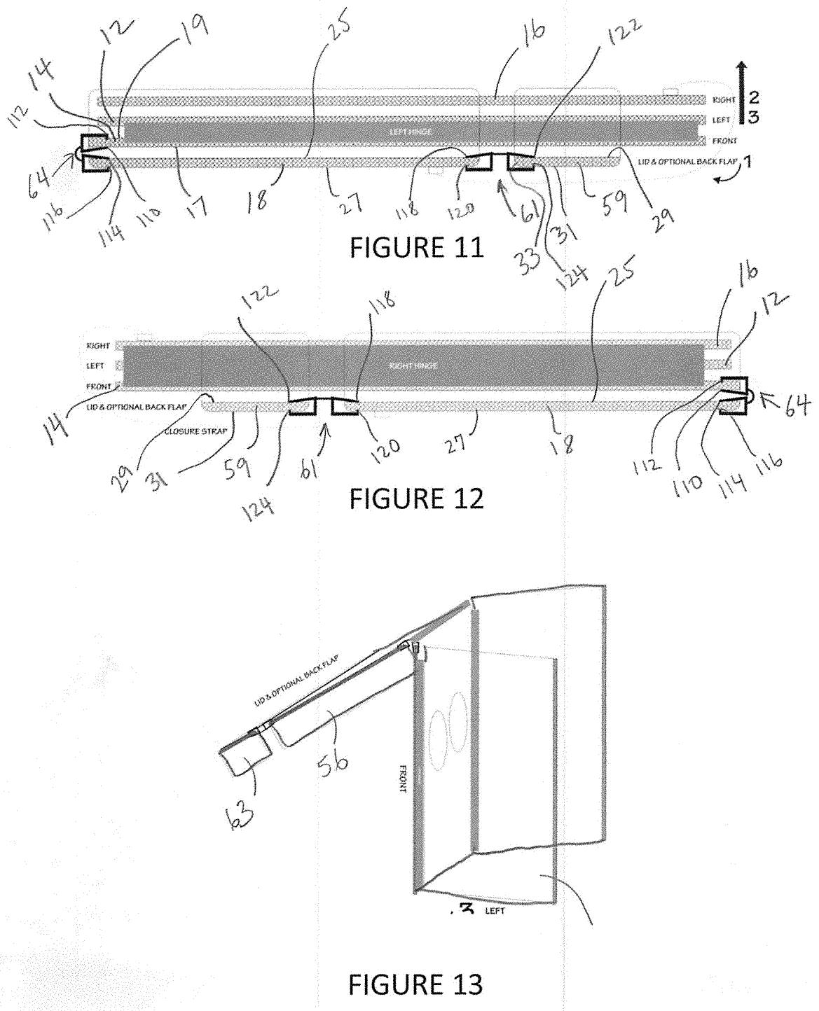

As shown in FIGS. 11 and 12, the front panel 14 includes a top inner longitudinal slot 110 on the inner surface 17 of the front panel 14 and adjacent the top edge 34 of the front panel 14. In a specific embodiment, the top inner longitudinal slot 110 is generally parallel to the top edge 34 of the front panel 14. A distal end of the left upper arm 76 on the hinge 64 is engaged with the top inner longitudinal slot 110 on the front panel 14. The front panel 14 includes a top outer longitudinal slot 112 on the outer surface 19 of the front panel 14 and adjacent the top edge 34 of the front panel 14. In a specific embodiment, the top outer longitudinal slot 112 is generally parallel to the top edge 34 of the front panel 14. The left lip 78 on the hinge 64 is engaged with the top outer longitudinal slot 112 on the front panel 14.

The top panel 18 includes a front inner longitudinal slot 114 on an inner surface 25 of the top panel 18 and adjacent the front edge 50 of the top panel 18. In a specific embodiment, the front inner longitudinal slot 114 is generally parallel to the front edge 50 of the top panel 18. A distal end of the right upper arm 84 on the top hinge 64 is engaged with the front inner longitudinal slot 114 on the top panel 18. The top panel 18 includes a front outer longitudinal slot 116 on an outer surface 27 of the top panel 18 and adjacent the front edge 50 of the top panel 18. In a specific embodiment, the front outer longitudinal slot 116 is generally parallel to the front edge 50 of the top panel 18. The right lip 86 on the top hinge 64 is engaged with the front outer longitudinal slot 116 on the top panel 18.

The top panel 18 includes a rear inner longitudinal slot 118 on the inner surface 25 of the top panel 18 and adjacent the rear edge 54 of the top panel 18. In a specific embodiment, the rear inner longitudinal slot 118 is generally parallel to the rear edge 54 of the top panel 18. A distal end of the left upper arm 76 on the rear hinge 61 is engaged with the rear inner longitudinal slot 118 on the top panel 18. The top panel 18 includes a rear outer longitudinal slot 120 on the outer surface 27 of the top panel 18 and adjacent the rear edge 54 of the top panel 18. In a specific embodiment, the rear outer longitudinal slot 120 is generally parallel to the rear edge 54 of the top panel 18. The left lip 78 on the rear hinge 61 is engaged with the rear outer longitudinal slot 120 on the top panel 18.

The back flap 59 includes a top inner longitudinal slot 122 on an inner surface 29 of the back flap 59 and adjacent a top edge 33 of the back flap 59. In a specific embodiment, the top inner longitudinal slot 122 is generally parallel to the top edge 33 of the back flap 59. A distal end of the right upper arm 84 on the rear hinge 61 is engaged with the top inner longitudinal slot 122 on the back flap 59. The back flap 59 includes a top outer longitudinal slot 124 on an outer surface 31 of the back flap 59 and adjacent the top edge 33 of the back flap 59. In a specific embodiment, the top outer longitudinal slot 124 is generally parallel to the top edge 33 of the back flap 59. The right lip 86 on the rear hinge 61 is engaged with the top outer longitudinal slot 124 on the top panel 18.

One of the characteristics of the living hinges 60 and 62, when attached to the left side panel 12, front panel 14, and right side panel 16 as shown in FIGS. 3 and 4, is that when the left side panel 12 and the right side panel 16 are rotated backwardly from their expanded position shown in FIG. 3 to their erected position shown in FIGS. 4 and 15, the hinges 60/62 are placed in tension and function to apply a reverse spring force to urge the left side panel 12 and the right side panel 16 back toward the expanded position as shown in FIG. 3. In use, once the left side panel 12 and the right side panel 16 are rotated into the erected position as shown in FIG. 4, the top panel 18 is then lowered down until it rests on the top edge 26 of the left side panel 12 and the top edge 42 of the right side panel 16, with the left lip member 56 on the top panel 18 engaged with the outer surface 15 of the left side panel 12, and the right lip member 58 on the top panel 18 engaged with the outer surface 23 of the right side member 16, as shown for example in FIGS. 1 and 15. When in this position, the spring force of the left hinge 60 urges the left side panel 12 against the left lip member 56 on the top panel 18, and the spring force of the right hinge 62 urges the right side panel 16 against the right lip member 58 on the top panel 18, which assists in holding the collapsible healthcare barrier device 10 in its erected position. In a preferred embodiment, to facilitate creation of these spring forces, the living hinges 60 and 62 are affixed to the left side panel 12, the front panel 14, and the right side panel 16 with central connectors 68 and the upper arms 76 and 84 disposed adjacent the outer surfaces 15, 19, and 23 of the left side panel 12, the front panel 14, and the right side panel 16, respectively.

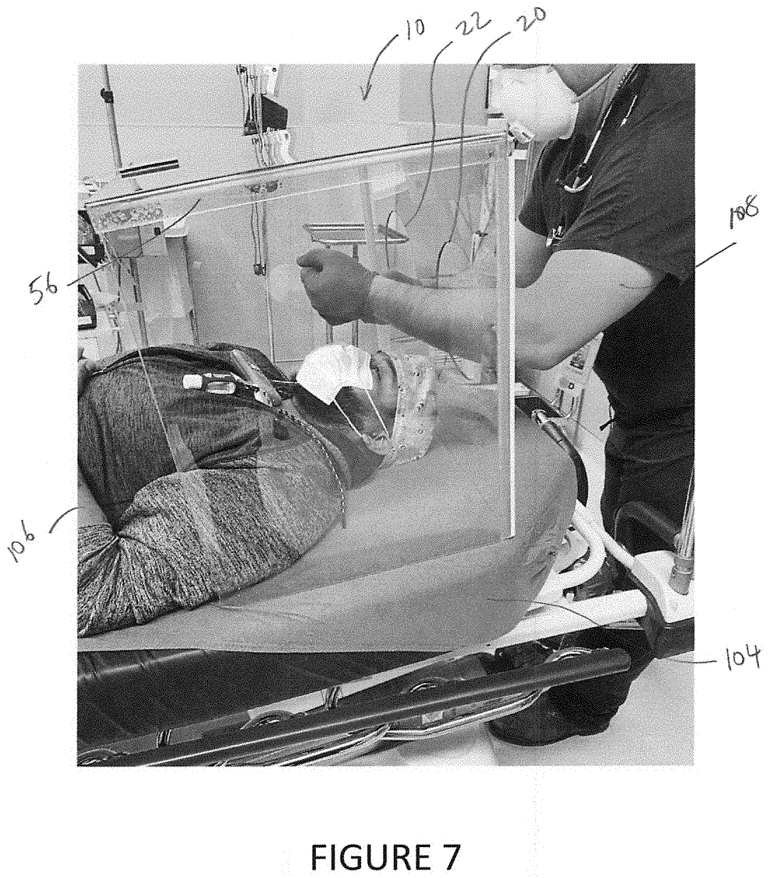

Referring now to FIG. 6, a specific embodiment of a collapsible healthcare barrier device 10 is shown in its erected configuration and positioned on a hospital bed 104 and over the head and chest of a patient 106. The first and second passageways 20 and 22 in the front panel 14 are positioned adjacent the head of the patient 106. With reference to FIG. 7, a health care provider 108 is shown with his hands and arms inserted through the first and second passageways 20 and 22 for purposes of rendering care to the patient 106.

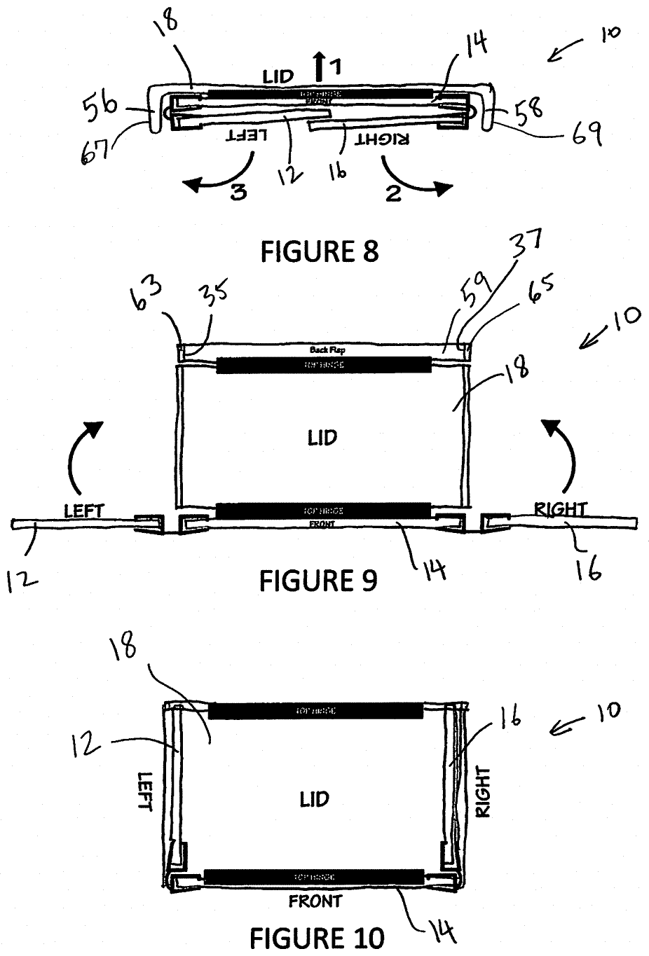

To collapse the collapsible healthcare barrier device 10 for storage, the back flap 59 is rotated upwardly into the position shown in FIG. 14. Next, the top panel 18 is lifted up to its expanded position shown in FIG. 2, which releases the left side panel 12 and the right side panel 16 to be rotated back to their expanded positions, as shown in FIG. 2. The left side panel 12 and the right side panel 16 are then rotated forward and folded down on top of the outer surface 19 of the front panel 14, as shown for example in FIG. 8. Either the left side panel 12 or the right side panel 16 is folded directly on top of the outer surface 19 of the front panel 14, and then the other one is folded down on top of the one directly on top of the front panel 14. The top panel 18 is rotated in the opposite direction and folded down on top of the inner surface 17 of the front panel 14. When this happens, as shown in FIG. 8, the collapsible healthcare barrier device 10 will be in its collapsed position with the left side panel 12, the front panel 14, and the right side panel 16 nested on top of the top panel 18 and between the left and right lip members 56 and 58 of the top panel 18. When in this configuration, the collapsible healthcare barrier device 10 is more portable than prior barrier boxes, and also can be stored in a space much smaller than the storage space required for prior barrier boxes.

It is to be understood that the inventions disclosed herein are not limited to the exact details of construction, operation, exact materials or embodiments shown and described. Although specific embodiments of the inventions have been described, various modifications, alterations, alternative constructions, and equivalents are also encompassed within the scope of the inventions. Although the present inventions may have been described using a particular series of steps, it should be apparent to those skilled in the art that the scope of the present inventions is not limited to the described series of steps. The specification and drawings are, accordingly, to be regarded in an illustrative rather than a restrictive sense. It will be evident that additions, subtractions, deletions, and other modifications and changes may be made thereunto without departing from the broader spirit and scope of the inventions as set forth in the claims set forth below. Accordingly, the inventions are therefore to be limited only by the scope of the appended claims.

1. A health care barrier device comprising:
a left side panel having a front edge, a top edge, a bottom edge, a rear edge, an inner surface, and an outer surface;
a front panel having a left edge, a top edge, a right edge, a bottom edge, at least one passageway, an inner surface, and an outer surface;
a right side panel having a front edge, a top edge, a bottom edge, a rear edge, an inner surface, and an outer surface;
a top panel having a left edge, a front edge, a right edge, and a rear edge;
the front edge of the left side panel being hingedly connected to the left edge of the front panel;
the front edge of the right side panel being hingedly connected to the right edge of the front panel;
the top edge of the front panel being hingedly connected to the front edge of the top panel; and
the left side panel, the front side panel, the right side panel, and the top panel being moveable between collapsed and erected positions.

2. The health care barrier device of claim 1, further including:
a left hinge connecting the left side panel to the front panel;
a right hinge connecting the right side panel to the front panel; and
a top hinge connecting the front panel to the top panel.

3. The health care barrier device of claim 1, further including a back flap having a top edge hingedly connected to the rear edge of the top panel, the back flap being moveable between a collapsed position and an erected position.

4. The health care barrier device of claim 3, further including a rear hinge connecting the back flap to the top panel.

5. The health care barrier device of claim 3, wherein the back flap further includes a left wing member extending from a left edge of the back flap and a right wing member extending from a right edge of the back flap.

6. The health care barrier device of claim 5, wherein the left wing member and the right wing member are disposed in generally perpendicular relationship with the back flap.

7. The health care barrier device of claim 1, wherein the top panel further includes a left lip member extending from the left edge of the top panel, and a right lip member extending from the right edge of the top panel, the left lip member being positioned adjacent the top edge and the outer surface of the left side panel when the top panel and the left side panel are in their erected positions, the right lip member being positioned adjacent the top edge and the outer surface of the right side panel when the top panel and the right side panel are in their erected positions.

8. The health care barrier device of claim 7, wherein the left lip member and the right lip member are disposed in generally perpendicular relationship with the top panel.

9. A health care barrier device comprising:
a left side panel having a front edge, a top edge, a bottom edge, a rear edge, an inner surface, and an outer surface;
a front panel having a left edge, a top edge, a right edge, a bottom edge, at least one passageway, an inner surface, and an outer surface;
a right side panel having a front edge, a top edge, a bottom edge, a rear edge, an inner surface, and an outer surface;
a top panel having a left edge, a front edge, a right edge, and a rear edge;
the front edge of the left side panel being hingedly connected to the left edge of the front panel;
the front edge of the right side panel being hingedly connected to the right edge of the front panel;
the top edge of the front panel being hingedly connected to the front edge of the top panel;
the left side panel, the front side panel, the right side panel, and the top panel being moveable between collapsed and erected positions; and,
the top panel further including a left lip member extending from the left edge of the top panel, and a right lip member extending from the right edge of the top panel, the left lip member being positioned adjacent the top edge and the outer surface of the left side panel when the top panel and the left side panel are in their erected positions, the right lip member being positioned adjacent the top edge and the outer surface of the right side panel when the top panel and the right side panel are in their erected positions.

10. The health care barrier device of claim 9, further including:
a left hinge connecting the left side panel to the front panel;
a right hinge connecting the right side panel to the front panel; and
a top hinge connecting the front panel to the top panel.

11. The health care barrier device of claim 9, wherein the left lip member and the right lip member are disposed in generally perpendicular relationship with the top panel.

12. The health care barrier device of claim 9, further including a back flap having a top edge hingedly connected to the rear edge of the top panel, the back flap being moveable between a collapsed position and an erected position.

13. The health care barrier device of claim 12, further including a rear hinge connecting the back flap to the top panel.

14. The health care barrier device of claim 12, wherein the back flap further includes a left wing member extending from a left edge of the back flap and a right wing member extending from a right edge of the back flap, the left wing member being positioned adjacent an outer surface of the left lip member when the top panel, the left side panel, and the back flap are in their erected positions, and the right wing member being positioned adjacent an outer surface of the right lip member when the top panel, the right side panel, and the back flap are in their erected positions.

15. The health care barrier device of claim 14, wherein the left wing member and the right wing member are disposed in generally perpendicular relationship with the back flap.

16. A health care barrier device comprising:
a left side panel having a front edge, a top edge, a bottom edge, a rear edge, an inner surface, and an outer surface;
a front panel having a left edge, a top edge, a right edge, a bottom edge, at least one passageway, an inner surface, and an outer surface;
a right side panel having a front edge, a top edge, a bottom edge, a rear edge, an inner surface, and an outer surface;
a top panel having a left edge, a front edge, a right edge, and a rear edge;
a back flap having a left edge, a top edge, and a right edge;
the front edge of the left side panel being hingedly connected to the left edge of the front panel;
the front edge of the right side panel being hingedly connected to the right edge of the front panel;
the top edge of the front panel being hingedly connected to the front edge of the top panel;
the top edge of the back flap being hingedly connected to the rear edge of the top panel,
the left side panel, the front side panel, the right side panel, the top panel, and the back flap being moveable between collapsed and erected positions; and,
the top panel further including a left lip member extending from the left edge of the top panel, and a right lip member extending from the right edge of the top panel, the left lip member being positioned adjacent the top edge and the outer surface of the left side panel when the top panel and the left side panel are in their erected positions, the right lip member being positioned adjacent the top edge and the outer surface of the right side panel when the top panel and the right side panel are in their erected positions.

17. The health care barrier device of claim 16, further including:
a left hinge connecting the left side panel to the front panel;
a right hinge connecting the right side panel to the front panel;
a top hinge connecting the front panel to the top panel; and
a rear hinge connecting the back flap to the top panel.

18. The health care barrier device of claim 16, wherein the left lip member and the right lip member are disposed in generally perpendicular relationship with the top panel.

19. The health care barrier device of claim 16, wherein the back flap further includes a left wing member extending from a left edge of the back flap and a right wing member extending from a right edge of the back flap, the left wing member being positioned adjacent an outer surface of the left lip member when the top panel, the left side panel, and the back flap are in their erected positions, and the right wing member being positioned adjacent an outer surface of the right lip member when the top panel, the right side panel, and the back flap are in their erected positions.

20. The health care barrier device of claim 19, wherein the left wing member and the right wing member are disposed in generally perpendicular relationship with the back flap.
